(19) United States
(12) Patent Application Publication
(10) Pub. No.: US 2022/0211627 A1
(43) Pub. Date: Jul. 7, 2022

(54) DRY MICROPARTICLES

(71) Applicant: UCB BIOPHARMA SRL, BRUSSELS (BE)

(72) Inventors: Audrey Françoise ARRIGHI, Brussels (BE); Jonathan Eleuthère Maurice GOOLE, Brussels (BE); Sarah MARQUETTE, Brussels (BE)

(21) Appl. No.: 17/595,176

(22) PCT Filed: May 13, 2020
(86) PCT No.: PCT/EP2020/063326

§ 371 Date: Nov. 10, 2021

(30) Foreign Application Priority Data
May 15, 2019 (GB) 1906835.2

(51) Int. Cl. A61K 9/16; A61K 9/19; A61K 39/395

(52) U.S. Cl. CPC A61K 9/1647; A61K 9/1652; A61K 2039/505; A61K 39/39591; A61K 9/19

(57) ABSTRACT

The present invention is directed to pharmaceutical compositions, and in particular to slow release pharmaceutical compositions comprising antibody molecule-loaded polymeric microspheres, in the form of dry microparticles. The dry microparticles, and pharmaceutical compositions comprising said dry microparticles, are stable during manufacturing and upon storage and demonstrate interesting slow-release characteristics. In addition, the invention relates to methods for preparing said dry microparticles.

### FIELD OF THE INVENTION

The present invention is directed to pharmaceutical compositions, and in particular to slow release pharmaceutical compositions comprising antibody molecule-loaded polymeric microspheres, in the form of dry microparticles. The dry microparticles, and pharmaceutical compositions comprising said dry microparticles, are stable during manufacturing and upon storage and demonstrate interesting slow-release characteristics. In addition, the invention relates to methods for preparing said dry microparticles.

### TECHNICAL BACKGROUND

Typically, therapeutic proteins such as antibodies are administered subcutaneously or intravenously. Nevertheless, patients and physicians may not be willing to use these drugs due to the pain and inconveniences if they are administered repeatedly by these invasive routes. Unfortunately, most of the therapeutic proteins on the market require frequent administration.

One formulation format that may improve the dosing regimen for a given drug is the sustained release (also known as slow-release) format: it allows the slow release of a drug usually encapsulated in a polymeric matrix, possibly over few months. Very often, in such a slow release formulation, an initial large amount of drug is released before a stable release profile is reached: this is called a burst release. The burst release leads to high initial drug delivery and possibly to adverse side effects.

Among the various sustained release formulation formats that are available, dry powder compositions, such as dry microparticle compositions, are well established. However, when it comes to their use for administering therapeutic proteins, they present some deficiencies. Indeed, proteins are often subject to aggregation and low extractability, strongly decreasing the efficiency of dry microparticle compositions. This is particularly true when the therapeutic protein formulated as a dry microparticle is an antibody molecule.

One method for preparing relatively stable dry microparticles containing therapeutic proteins is spray-drying. It is a process converting a liquid-based formulation into a dry powder by atomizing the liquid formulation in droplets, into a hot drying-medium, typically air or nitrogen. The process provides enhanced control over particle size, size distribution, particle shape, density, purity and structure. Compositions to be spray-dried generally comprise polyols. Nevertheless, this technique has some drawbacks such as agglomeration issues and the low yields that are obtained due to the adhesion of the particles to the inner walls of the spray-drying apparatus.

The starting material for spray-drying is typically an emulsion. Double emulsion techniques (e.g. water-in-oil-in-water (WOW), solid-in-oil-in-water(SOW)) are commonly used to produce protein-loaded Poly(lactide-co-glycolide) Acid (PLGA) microparticles with sustained-release properties. However, a significant amount of protein may be lost into the external aqueous phase, leading to a significant decrease of the drug loading (DL) (Wang J. et al., 2004). The spray-drying of a water-in-oil (w/o) emulsion seems to be a suitable alternative to produce protein-loaded microparticles. Indeed, spray-drying is a one-step process that is reproducible and easily scalable. Moreover, compared to double emulsion techniques, the spray-drying of a w/o emulsion avoids the presence of an external aqueous phase which may lead to the production of microparticles with higher DL (Giunchedi et al., 2001). This approach has been successfully used to produce high protein-loaded microparticles with sustained-release properties, using polyclonal immunoglobulin G as an antibody model. Nevertheless, when this process was applied to a monoclonal antibody (mAb), stability issues were observed through the formation of High Molecular Weight Species (HMWS) during the encapsulation process. Surface induced aggregation (contact of the mAb with the organic phase) was hypothesized as the main cause of mAb instability. These HMWS should be avoided since they can induce immunogenicity, thus affecting the safety and efficacy of the product (Moussa et al., 2016).

For any kind of formulation (liquid, freeze-dried, spray-dried, etc), non-ionic surfactants such as polysorbate 20, polysorbate 80, poloxamer 188 are usually used for mAb stabilization against surface-induced aggregation. However, this type of surfactants and more particularly the polyoxyethylene-based surfactants show several disadvantages such as stability issues during long-term storage due to the formation of mixed micelles with proteins. In this context, cyclodextrins have emerged as alternative excipients for this purpose for instance (Pai et al., 2009; Serno et al., 2010; U.S. Pat. No. 5,997,856). Nevertheless, when used in spray-dried formulations, cyclodextrins did not have the expected performance nor the expected stability effects on proteins (Johansen et al., 1998). Further, it has some disadvantages such as its adsorption of water.

Other aspects to consider with slow-released compositions are the encapsulation efficiency, drug loading and their effect on initial “burst release” (Han et al., 2016).

Therefore, there remains a need for further pharmaceutical compositions comprising antibody-loaded polymeric microspheres (provided as dry microparticles) with sustained-release properties, improving stability of antibodies (e.g. limiting antibody degradation during the production of antibody-loaded polymeric microspheres by spray-drying a water-in-oil emulsion), while providing good powder performance (e.g. high encapsulation efficiency at high drug loading, high extraction efficiency and acceptable initial burst release).

### SUMMARY OF THE INVENTION

The present invention addresses the above needs by providing a dry antibody molecule-loaded polymeric microsphere (alternatively named dry microparticle) comprising an antibody molecule, a polymer and cyclodextrin and optionally further comprising a buffering agent and/or a surfactant. Preferably, the cyclodextrin is a member of the β-cyclodextrin family, even more preferably selected from the group consisting of HPβCD and SBEβCD. Alternatively, it can also be a member of the α-cyclodextrin family. The dry microparticle (or the dry microparticles in its plural form) according to the invention can be resuspended before being administered to the patient in need thereof.

Also provided is an aqueous antibody molecule-containing emulsion comprising an antibody molecule, a polymer and cyclodextrin and optionally comprising a buffering agent and/or a surfactant. Preferably, the cyclodextrin is a member of the β-cyclodextrin family, even more preferably selected from the group consisting of HPβCD and SBEβCD. Alternatively, it can also be a member of the α-cyclodextrin family. Said aqueous antibody molecule-containing emulsion can be used to produce, by spray-drying, a dry microparticle.

Also encompassed is a pharmaceutical composition comprising the dry microparticle(s) according to the invention.

In the context of the invention as a whole, the antibody molecule is selected from the group consisting of a complete antibody molecule having full length heavy and light chains, or an antigen-binding fragment thereof, for example selected from the group consisting of (but not limited to) Fab, modified Fab, Fab′, modified Fab′, F(ab′)2, Fv, Fab-Fv, Fab-dsFv, Fab-Fv-Fv, scFv, Bis-scFv fragment, Fab linked to one or two scFvs or dsscFvs, such as BYbe® or a TRYbe®, diabody, tribody, triabody, tetrabody, minibody, single domain antibody, camelid antibody, Nanobody™ or VNAR fragment.

In one aspect, here are provided aqueous antibody molecule-containing emulsions and dry microparticles comprising an antibody molecule, a polymer, and cyclodextrin, wherein the antibody molecule/cyclodextrin ratio (w/w) is from 12:1 to 7:6.

A method for producing the dry microparticle according to the invention is also provided, as well as a process for obtaining said dry microparticle, a method for stabilizing an antibody molecule in said dry microparticle and a method for improving the sustained release performance of said dry microparticle.

### DETAILED DESCRIPTION

The term “solvent”, as used herein, refers to a liquid solvent either aqueous or non-aqueous.

When the solvent is used for resuspending a drug compound, the selection of the solvent depends notably on the solubility of the drug compound on said solvent and on the mode of administration. For resuspending microparticles comprising a protein, such as an antibody, aqueous solvents are preferred. Aqueous solvent may consist solely of water, or may consist of water plus one or more miscible solvents, and may contain dissolved solutes such as buffers, salts or other excipients. According to the present invention, the preferred solvent for resuspending the one or more microparticles before administration to a patient is an aqueous solvent such as water or a saline solvent.

When the solvent is used for solubilising the polymer needed for forming the antibody-loaded microspheres, it is typically selected from the group consisting of acetonitrile, ethyl acetate, acetone, tetrahydrofuran and chlorinated solvents (such as dichloromethane).
The term “dry microparticle” (dry microparticles in its plural form) refers to a dry “particle” of very small size (size typically of about 20 μm or below) (alternatively named “microparticles” or “microspheres”). Preferably the dry microparticle contains water below about 10%, usually below 5% or even below 3% by weight of the dry particles. Said dry microparticle corresponds to the dried antibody-loaded microsphere (alternatively named microsphere or MS) in the context of the present invention. A dry microparticle can typically be obtained by spray-drying and/or freeze-drying an aqueous solution or an aqueous emulsion. Alternatively, the term dry powder can be used.
The term “aqueous antibody molecule-containing emulsion” refers to a water-in-oil-in-water or to a water-in-oil emulsion and is further defined herewith. In the context of the present invention, a water-in-oil emulsion is preferred.
The term “freeze-drying” also known as “lyophilization” refers to a process for obtaining a dry microparticle consisting of at least three main steps: 1) lowering the temperature of the product to be freeze-dried to below freezing point (typically between −40 and −80° C.; freezing step), 2) high-pressure vacuum (typically between 30 and 300 mTorr; first drying step) and 3) increasing the temperature (typically between 20 and 40° C.; second drying step).
The term “spray drying” refers to a process for obtaining a dry microparticle consisting of at least two main steps: 1) atomizing a liquid feed into fine droplets and 2) evaporating the solvent or water by means of a hot drying gas.
The term “slow-release” (herein alternatively named “sustained-release”) refers to the delivery of the active ingredient (such as an antibody or an antigen-binding fragment thereof) over days, weeks, months or even years. The typical slow-release profile for a protein-loaded PLGA microparticle is triphasic and consists of (i) an initial burst release (i.e. the release of an initial large amount of active ingredient), (ii) a lag phase (i.e. a phase during which very low amount or no product is released) and (iii) a release phase (i.e. a phase during which the release rate is stable) (Diwan et al., 2001 and White et al., 2013). An initial burst release of preferably no more than about 50% of the total amount of active ingredient will be deemed acceptable. Any initial burst release of no more than 40% will be called a “limited burst release”. The release of the antibody molecule should also be as complete as possible (i.e. total release as close as possible to 100% of the encapsulated antibodies), and preferably at least above 90%. One of the advantages of such a slow-release composition is that the composition will be administered less often to the patient.
The term “stability”, as used herein, refers to the physical, chemical, and conformational stability of the antibody molecule in the compositions according to the present invention (and including maintenance of biological potency). Instability of an antibody molecule formulation may be caused by chemical degradation or aggregation of the antibody molecules to form for instance higher order polymers, deglycosylation, modification of glycosylation, oxidation or any other structural modification that reduces the biological activity of the formulated antibody molecules.
The term “stable” (such as in “stable dry microparticle”) refers to a microparticle or a pharmaceutical composition in which the antibody molecule of interest essentially retains its physical, chemical and/or biological properties during manufacturing and upon storage. In order to measure the antibody molecule stability in a formulation, various analytical methods are well within the knowledge of the skilled person (see some examples in the example section). Various parameters can be measured to determine stability (in comparison with the initial data), such as (and not limited to): 1) no more than 10% of alteration of the monomeric form of the antibody, or 2) no more than 5% of increase in High Molecular Weight Species (HMW or HMWS; also herein referred to as aggregates).
The term “buffer” or “buffering agent”, as used herein, refers to solutions of compounds that are known to be safe in formulations for pharmaceutical use and that have the effect of maintaining or controlling the pH of the formulation in the pH range desired for the formulation. Acceptable buffers for controlling pH at a moderately acidic pH to a moderately basic pH include, but are not limited to, phosphate, acetate, citrate, arginine, TRIS (2-amino-2-hydroxymethyl-1,3,-propanediol), histidine buffers and any pharmacologically acceptable salt thereof.
The term “surfactant”, as used herein, refers to a soluble compound that can be used notably to increase the water solubility of hydrophobic, oily substances or otherwise increase the miscibility of two substances with different hydrophobicity. Surfactants are commonly used in formulations, notably in order to modify the absorption of the drug or its delivery to the target tissues. Well known surfactants include polysorbates (polyoxyethylene derivatives; Tween) as well as poloxamers (i.e. copolymers based on ethylene oxide and propylene oxide, also known as Pluronics®). According to the invention, the preferred surfactant is a poloxamer surfactant and even more preferably is poloxamer 407 (also known as Pluronic® F127).
The term “stabilizing agent”, “stabilizer” or “isotonicity agent”, as used herein, is a compound that is physiologically tolerated and imparts a suitable stability/tonicity to a formulation. During freeze-drying (lyophilization) process or spray drying process, the stabilizer is also effective as a protectant. Compounds such as glycerin, are commonly used for such purposes. Other suitable stabilizing agents include, but are not limited to, amino acids or proteins (e.g. glycine or albumin), salts (e.g. sodium chloride), and sugars (e.g. dextrose, mannitol, sucrose, trehalose and lactose). According to the present invention, the preferred stabilizing agent is a cyclodextrin.
The term “cyclodextrin” (or its plural form) is a compound consisting of several glucose subunits (6 to 8), arranged such as to form a ring. Cyclodextrins are widely accepted in liquid compositions for parenteral use in humans. The preferred form of cyclodextrin according to the invention belongs to the β-cyclodextrin family (7-glucose subunits), such as (but not limited to) hydroxypropyl-β-cyclodextrin (HPβCD) and sulfobutyl ether β-cyclodextrin (SBEβCD). Alternatively, a member of the α-cyclodextrin family (6-glucose subunits) could be used, but preferably not a γ-cyclodextrin (8-glucose subunits).
The term “polymer” refers to a high molecular weight polymeric compound or macromolecule built by the repetition of simple chemical units. A polymer may be a biological polymer, naturally occurring (e. g., proteins, carbohydrates, nucleic acids) or a synthetically-produced polymer (such as polyethylene glycols, polyvinylpyrrolidones). The term polymer also includes copolymers. Biodegradable and biocompatible polymers are preferred in the context of the present invention. Examples of such polymers (or co-polymers) are polylactic acid (PLA), copolymers of PLA with glycolic acid (PLGA), PEGylated PLGA or yet polycaprolactone PCL.
The term “vial” or “container”, as used herein, refers broadly to a reservoir suitable for retaining the pharmaceutical compositions of the invention as dry microparticles. Similarly, it will retain the solvent for resuspension, if needed. Examples of a vial that can be used in the present invention include (but not limited to) syringes (such as a pre-filled syringe), ampoules, cartridges, tubes, bottles or other such reservoirs suitable for storage and/or delivery of the pharmaceutical composition to the patient. The vial may be part of a kit-of-parts comprising one or more containers comprising the pharmaceutical compositions according to the invention and delivery devices such as a syringe, pre-filled syringe, an autoinjector, a needleless device, an implant or a patch, or other devices for parental administration and instructions of use.
The term “antibody molecule” means a complete antibody molecule having full length heavy and light chains, or an antigen-binding fragment thereof. An antigen-binding fragment can be selected, for example, from the group comprising or consisting of (but not limited to) a Fab, modified Fab, Fab′, modified Fab′, F(ab′)2, Fv, Fab-Fv, Fab-dsFv, Fab-Fv-Fv, scFv and Bis-scFv fragment. Said fragment can also be a diabody, tribody, triabody, tetrabody, minibody, single domain antibody (dAb) such as sdAb, VL, VH, VHH or camelid antibody (e.g. from camels or llamas such as a Nanobody™) and VNAR fragment. An antigen-binding fragment according to the invention can also comprise a Fab linked to one or two scFvs or dsscFvs, each scFv or dsscFv binding the same or a different target (e.g., one scFv or dsscFv binding a therapeutic target and one scFv or dsscFv that increases half-life by binding, for instance, albumin). Exemplary of such antibody fragments are FabdsscFv (also referred to as BYbe®) or Fab-(dsscFv)₂ (also referred to as TrYbe®, see WO2015/191172 for instance). The antibody molecule according to the invention can be a mono, bi, tri or tetra-valent, bispecific, trispecific, tetraspecific or multispecific antibody molecule formed from antibodies or antibody fragments. The term includes antibody molecules of any species, in particular of mammalian species, having two essentially complete heavy and two essentially complete light chains, human antibodies of any isotype, including IgA1, IgA2, IgD, IgG1, IgG2a, IgG2b, IgG3, IgG4, IgE and IgM and modified variants thereof, non-human primate antibodies, e.g. from chimpanzee, baboon, rhesus or cynomolgus monkey, rodent antibodies, e.g. from mouse, rat or rabbit; goat or horse antibodies, and derivatives thereof, or of bird species such as chicken antibodies or of fish species such as shark antibodies. Said antibody molecules can be of any types such as monoclonal, chimeric, humanized, fully-human. If desired, an antibody molecule may be conjugated to one or more effector molecule(s). Antibody molecules as defined above are well known in the art as well as methods for creating and manufacturing these antibodies or antibody fragments (Verma et al., 1998).

The antibody or antigen-binding fragment thereof can be obtained by culturing prokaryotic or eukaryotic host cells transfected with one or more expression vectors encoding the recombinant antibody or recombinant antibody fragment(s). The eukaryotic host cells are preferably mammalian cells, more preferably Chinese Hamster Ovary (CHO) cells. The prokaryotic host cells are preferably gram-negative bacteria, more preferably, the host cells are E. coli cells. The host cells may be cultured in any medium that will support their growth and expression of the recombinant protein. The best conditions for each host cell would be known to those skilled in the art. Once recovered either from the supernatant of a cell culture or from inclusion bodies, depending on the host cell used for the production, the antibody or antigen-binging fragment thereof can be purified. Purification methods are well-known to those skilled in the art. They typically consist of a combination of various chromatographic and filtration steps. The full process is performed in aqueous condition. The solution recovered at the end of the process can be submitted to formulation. Said solution will herein be called “aqueous antibody molecule-containing solution”. It refers to the solution from which the emulsion and then the dry microparticle(s) of the invention are formed.
The term “high concentration” antibody molecule means that the concentration of antibody molecule is at least 50 mg/mL.
The term “therapeutically effective amount” as used herein refers to the amount of an antibody molecule needed to treat, ameliorate or prevent a targeted disease, disorder or condition, or to exhibit a detectable therapeutic, pharmacological or preventative effect. For any antibody molecule, the therapeutically effective amount can be estimated initially either in cell culture assays or in animal models, usually in rodents, rabbits, dogs, pigs or primates. The animal model may also be used to determine the appropriate concentration range and route of administration. Such information can then be used to determine useful doses and routes for administration in humans. In all the embodiments of the present invention, “composition” can also be referred to as “formulation” without any differentiation.

It was a surprising finding of the inventors that some properties of pharmaceutical compositions in the form of dry microparticles were deeply improved in presence of cyclodextrin, and more especially in presence of some members of the β-cyclodextrin family, such as HPβCD and SBEβCD. These effects were in particular observed with a dry microparticle (or dry microparticles) obtained from an aqueous solution comprising the antibody molecules at high concentration and when the spray drying step was performed with emulsions. It was indeed surprisingly found that the dry microparticle(s) according to the invention had sustained-release properties and improved the stability of antibodies, while providing good powder performance (e.g. high encapsulation efficiency at high drug loading, high extraction efficiency and acceptable initial burst release).

In the context of the invention, the dry microparticle will be considered as having good powder performance should it present an encapsulation efficiency above 90%, a drug loading above 20% and an extraction efficiency above 80%. An increase of at least about 10% of the total amount of mAb released would be considered as an improvement from a powder performance. A decrease of at least 10% of the HMWS, compared to a formulation containing no cyclodextrin, would be considered as an improvement from a stability viewpoint.

The main object of the present invention is a dry microparticle comprising or consisting of an antibody molecule, a polymer, and cyclodextrin. Optionally, said dry microparticle further comprises a buffering agent and/or a surfactant. As an example, herein is provided a dry microparticle comprising or consisting of about 10 to 30% weight (w)/w of an antibody molecule, about 50 to 80% (w/w) of a polymer, a cyclodextrin in an antibody molecule/cyclodextrin ratio (w/w) of from or from about 12:1 to or to about 7:6 and optionally about 0.2 to 4% (w/w) of a buffering agent, and/or about 0.05 to 4.0% (w/w) of a surfactant. As a further example, herein is provided a dry microparticle comprising or consisting of about 10 to 30% (w/w) of an antibody molecule, about 0.2 to 4% (w/w) of a buffering agent, about 50 to 80% (w/w) of a polymer, a cyclodextrin in an antibody molecule/cyclodextrin ratio (w/w) of from or from about 12:1 to or to about 7:6 and optionally about 0.05 to 4.0% (w/w) of a surfactant. Said microparticle is stable. It is understood that in any case the sum of the percentages of all the components reaches 100%.

Another object of the present invention is an aqueous antibody molecule-containing emulsion comprising or consisting of an antibody molecule, a polymer, and cyclodextrin. Optionally, said aqueous antibody molecule-containing emulsion further comprises a buffering agent and/or a surfactant. As an example, herein is provided an aqueous antibody molecule-containing emulsion comprising or consisting of: a) an aqueous phase comprising or consisting of about 5 to about 30% w/v (weight/volume) (i.e. about 50 to about 300 mg/mL) of an antibody molecule, a cyclodextrin in an antibody molecule/cyclodextrin ratio (w/w) of from or from about 12:1 to or to about 7:6 and optionally about 5 to 100 mM of a buffering agent and about 0.05 to about 1.5% w/v of a surfactant and b) an organic phase comprising about 0.5 to about 10.0% w/v of a polymer. Expressed in w/w, the aqueous antibody molecule-containing emulsion herein provided comprises or consists of about 10 to 30% (w/w) of an antibody molecule, about 50 to 80% (w/w) of a polymer, a cyclodextrin in an antibody molecule/cyclodextrin ratio (w/w) of from or from about 12:1 to or to about 7:6 and optionally about 0.2 to 4% (w/w) of a buffering agent, and/or about 0.05 to 4.0% (w/w) of a surfactant. As a further example, herein is provided an aqueous antibody molecule-containing emulsion comprising or consisting of about 10 to 30% (w/w) of an antibody molecule, about 0.2 to 4% (w/w) of a buffering agent, about 50 to 80% (w/w) of a polymer, a cyclodextrin in an antibody molecule/cyclodextrin ratio (w/w) of from or from about 12:1 to or to about 7:6 and optionally about 0.05 to 4.0% (w/w) of a surfactant. Said aqueous antibody molecule-containing emulsion can be used as an intermediate to obtain a dry microparticle by any known means. Preferably, said aqueous antibody molecule-containing emulsion can be spray-dried to obtain a dry microparticle. Alternatively, it can be first spray-dried and then freeze-dried to obtain a dry microparticle.

Another object of the present invention is a dry microparticle which is obtained by spray-drying an aqueous antibody molecule-containing emulsion. Said emulsion is obtained by homogenizing an aqueous phase and an organic phase and comprises or consists of a polymer (provided by the organic phase) and an antibody molecule, a cyclodextrin and optionally a buffering agent and/or a surfactant (provided by the aqueous phase). As an example, herein is provided a dry microparticle obtained by spray-drying an aqueous antibody molecule-containing emulsion, wherein said aqueous antibody molecule-containing emulsion comprises or consists of: a) an aqueous phase comprising or consisting of about 5 to about 30% w/v (i.e. about 50 to about 300 mg/mL) of an antibody molecule, a cyclodextrin in an antibody molecule/cyclodextrin ratio (w/w) of from or from about 12:1 to or to about 7:6 and optionally about 5 to 100 mM of a buffering agent and about 0.05 to about 1.5% w/v of a surfactant and b) an organic phase comprising about 0.5 to about 10.0% w/v of a polymer. As a further example, herein is provided a dry microparticle obtained by spray-drying an aqueous antibody molecule-containing emulsion, wherein said aqueous antibody molecule-containing emulsion comprises or consists of: a) an aqueous phase comprising or consisting of about 5 to about 30% w/v (i.e. about 50 to about 300 mg/mL) of an antibody molecule, about 5 to 100 mM of a buffering agent, a cyclodextrin in an antibody molecule/cyclodextrin ratio (w/w) of from or from about 12:1 to or to about 7:6 and optionally about 0.05 to about 1.5% w/v of a surfactant and b) an organic phase comprising about 0.5 to about 10.0% w/v of a polymer. After the step of spray-drying, the dry microparticle may optionally be further freeze-dried. Said microparticle is stable.

It is a further object of the present disclosure to describe a method for producing a dry microparticle comprising or consisting of an antibody molecule, a polymer, a cyclodextrin and optionally a buffering agent and/or a surfactant, said method comprising the steps of:
a) adding cyclodextrin, to an aqueous antibody molecule-containing solution to obtain an aqueous phase,
b) solubilising the polymer in a solvent, to obtain an organic phase,
c) adding the aqueous phase of step a) to the organic phase of step b) to obtain an aqueous antibody molecule-containing emulsion (after homogenization), and then
d) spray-drying the aqueous antibody molecule-containing emulsion to obtain the dry microparticle, and
e) optionally further freeze-drying the dry microparticle of step d) to obtain the final dry microparticle,
wherein steps a) and b) can be performed in any order.

Should the microparticle comprise a buffering agent and/or a surfactant, said buffering agent and/or surfactant is/are preferably present in the aqueous antibody molecule-containing solution (of step a). As an example, herein is disclosed a method for producing a dry microparticle comprising or consisting of an antibody molecule, a polymer, a cyclodextrin and optionally a buffer and/or a surfactant, said method comprising the steps of:
a) adding cyclodextrin at an antibody molecule/cyclodextrin ratio (w/w) of from or from about 12:1 to or to about 7:6 to an aqueous antibody molecule-containing solution (comprising about 5 to about 30% w/v (i.e. about 50 to about 300 mg/mL) of the antibody molecule), to obtain an aqueous phase,
b) solubilising the polymer in a solvent, to obtain an organic phase,
c) adding the aqueous phase of step a) to the organic phase of step b) comprising about 0.5 to about 10.0% w/v of polymer, to obtain an aqueous antibody molecule-containing emulsion (after homogenization), and then,
d) spray-drying aqueous antibody molecule-containing emulsion of step c) to obtain the dry microparticle, and
e) optionally further freeze-drying the dry microparticle of step d) to obtain the final dry microparticle,
wherein steps a) and b) can be performed in any order.

Should the microparticle comprise a buffering agent, said buffering agent is preferably present in the aqueous antibody molecule-containing solution (of step a) in an amount of about 5 to 100 mM of the buffering agent. Should the microparticle comprise a surfactant, said surfactant is preferably added (during step a) or before step a)) in the aqueous antibody molecule-containing solution at about 0.05 to about 1.5% w/v. As a further example, herein disclosed is a method for producing a dry microparticle comprising or consisting of an antibody molecule, a polymer, a cyclodextrin, a buffering agent and optionally a surfactant, said method comprising the steps of:
a) adding cyclodextrin at an antibody molecule/cyclodextrin ratio of from or from about 12:1 to or to about 7:6 (w/w) to an antibody molecule-containing solution comprising about 5 to about 30% w/v (i.e. about 50 to about 300 mg/mL) of the antibody molecule and about 5 to 100 mM of a buffering agent, to obtain an aqueous phase,
b) solubilising the polymer in a solvent, to obtain an organic phase,
c) adding the aqueous phase of step a) to the organic phase of step b) comprising about 0.5 to about 10.0% w/v of the polymer to obtain an aqueous antibody molecule-containing emulsion (after homogenization), and then
d) spray-drying the aqueous antibody molecule-containing emulsion of step c) to obtain the dry microparticle, and
e) optionally further freeze-drying the dry microparticle of step d) to obtain the final dry microparticle,
   wherein steps a) and b) can be performed in any order.

Should the microparticle comprise a surfactant, said surfactant is preferably added (during step a) or before step a)) in the aqueous antibody molecule-containing solution at about 0.05 to about 1.5% w/v.

Another aspect of the present invention is to provide a method for stabilizing an antibody molecule in a dry microparticle comprising the steps of: a) adding a cyclodextrin and then a solubilised polymer to an aqueous antibody molecule-containing solution, to obtain an aqueous antibody molecule-containing emulsion (after homogenisation) and then b) spray-drying the resulting aqueous antibody molecule-containing emulsion to obtain the dry microparticle in which the antibody molecule is stable. Should the microparticle comprise a buffering agent, said buffering agent is preferably present in the aqueous antibody molecule-containing solution (step a). As an example, herein is provided a method for stabilizing an antibody molecule in a dry microparticle comprising the steps of: a) adding a cyclodextrin at an antibody molecule/cyclodextrin ratio (w/w) of from or from about 12:1 to or to about 7:6 and then about 0.5 to about 10.0% w/v of a solubilised polymer, to an aqueous antibody molecule-containing solution (comprising about 5 to about 30% w/v (i.e. about 50 to about 300 mg/mL) of the antibody molecule), to obtain an aqueous antibody molecule-containing emulsion (after homogenisation) and then b) spray-drying the resulting aqueous antibody molecule-containing emulsion to obtain the dry microparticle in which the antibody molecule is stable. In another example, herein is provided a method for stabilizing an antibody molecule in a dry microparticle comprising the steps of: a) adding a cyclodextrin at an antibody molecule/cyclodextrin ratio (w/w) of from or from about 12:1 to or to about 7:6 and then about 0.5 to about 10.0% w/v of a solubilised polymer to an aqueous antibody molecule-containing solution (comprising about 5 to about 30% w/v (i.e. about 50 to about 300 mg/mL) of the antibody molecule and about 5 to 100 mM of a buffering agent), to obtain an aqueous antibody molecule-containing emulsion (after homogenisation) and then b) spray-drying the resulting aqueous antibody molecule-containing emulsion to obtain the dry microparticle in which the antibody molecule is stable. It is noted that should the microparticle comprise a surfactant, said surfactant is preferably added (during step a) or before step a)) in the aqueous antibody molecule-containing solution at about 0.05 to about 1.5% w/v. It is further noted that after the step of spray-drying, the dry microparticle may be further subjected to a step of freeze-drying.

Also described is a process for obtaining a dry microparticle comprising an antibody molecule, a polymer, a cyclodextrin and optionally a buffer and/or surfactant, comprising the steps of:
a. Adding cyclodextrin to an aqueous antibody molecule-containing solution to obtain a first composition (which is an aqueous phase),
b. combining the first composition of step a. to the polymer, wherein said polymer is solubilized (which is an organic phase), to obtain a second composition,
c. Homogenising the second composition of step b. to obtain a water-in-oil emulsion,
d. Spray-drying the water-in-oil emulsion of step c. to obtain said dry microparticle,
e. Optionally freeze-drying the dry microparticle of step d. to obtain the final dry microparticle.

Should the microparticle comprise a buffering agent and/or a surfactant, said buffering agent and/or surfactant is/are preferably present in the aqueous phase (step a). As an example, herein disclosed is a process for obtaining a dry microparticle comprising an antibody molecule, a polymer, a cyclodextrin and optionally a buffer and/or a surfactant, comprising the steps of:
a. Adding cyclodextrin at an antibody molecule/cyclodextrin ratio (w/w) of from or from about 12:1 to or to about 7:6 to an aqueous antibody molecule-containing solution (comprising about 5 to about 30% w/v (i.e. about 50 to about 300 mg/mL) of the antibody molecule) to obtain a first composition (which is an aqueous phase),
b. combining the first composition of step a. to about 0.5 to about 10.0% w/v of the polymer, wherein said polymer is solubilized (as an organic phase), to obtain a second composition,
c. Homogenising the second composition of step b. to obtain a water-in-oil emulsion,
d. Spray-drying the water-in-oil emulsion of step c. to obtain said dry microparticle,
e. Optionally freeze-drying the dry microparticle of step d. to obtain the final dry microparticle.

Should the microparticle comprise a buffering agent, said buffering agent is preferably present in the aqueous phase (step a) preferably in an amount of about 5 to 100 mM. Should the microparticle comprise a surfactant, said surfactant is also preferably added (during step a) or before step a)) in the aqueous phase at about 0.05 to about 1.5% w/v.

Alternatively, herein described is a process for obtaining a dry microparticle comprising an antibody molecule, a polymer, a cyclodextrin and optionally a buffer and/or a surfactant, comprises the steps of:
a. Adding cyclodextrin and then a solubilized polymer, to an aqueous antibody molecule-containing solution to obtain a first composition,
b. Homogenising the first composition of step a. to obtain a water-in-oil emulsion,
c. Spray-drying the water-in-oil emulsion of step b. to obtain said dry microparticle,
d. Optionally freeze-drying the dry microparticle of step c. to obtain the final dry microparticle.
   Should the microparticle comprise a buffering agent and/or a surfactant, said buffering agent and/or surfactant is/are preferably present in the aqueous antibody molecule-containing solution of step a. As an example, herein described is a process for obtaining a dry microparticle comprising an antibody, a polymer, a cyclodextrin and optionally a surfactant, comprising the steps of:
a. Adding cyclodextrin (at an antibody molecule/cyclodextrin ratio (w/w) of from or from about 12:1 to or to about 7:6) and then a solubilized polymer (at about 0.5 to about 10.0% w/v), to an aqueous antibody molecule-containing solution (comprising about 5 to about 30% w/v (i.e. about 50 to about 300 mg/mL) of the antibody molecule) to obtain a first composition,
b. Homogenising the first composition of step a. to obtain a water-in-oil emulsion,
c. Spray-drying the water-in-oil emulsion of step b. to obtain said dry microparticle,
d. Optionally freeze-drying the dry microparticle of step d. to obtain the final dry microparticle.
   Should the microparticle comprise a buffering agent, said buffering agent is preferably present in the aqueous antibody molecule-containing solution of step a. in an amount of about 5 to 100 mM of the buffering agent. Should the microparticle comprise a surfactant, said surfactant is also preferably added (during step a) or before step a)) in the aqueous antibody molecule-containing solution of step a, at about 0.05 to about 1.5% w/v.

Another object of the present invention is a method for improving the antibody molecule-sustained release performance of a dry microparticle, presenting for instance a limited burst release upon injection and/or a better total release of the antibody molecule, said method comprising the steps of: a) adding a cyclodextrin and then a solubilised polymer to an aqueous antibody molecule-containing solution, to obtain an aqueous antibody molecule-containing emulsion and then 2) spray-drying the resulting aqueous antibody molecule-containing emulsion, to obtain said dry microparticle with enhanced antibody molecule-sustained release performance. Should the microparticle comprise a buffering agent and/or a surfactant, said buffering agent and/or surfactant is/are preferably added in the aqueous antibody molecule-containing solution. As an example, herein is provided a method for enhancing the antibody molecule-sustained release performance of a dry microparticle, presenting a limited burst release upon injection and/or a better total release of the antibody molecule, said method comprising the steps of: a) adding cyclodextrin at an antibody molecule/cyclodextrin ratio (w/w) of from or from about 12:1 to or to about 7:6 and then about 0.5 to about 10.0% w/v of a solubilised polymer to an aqueous antibody molecule-containing solution (comprising about 5 to about 30% w/v (i.e. about 50 to about 300 mg/mL) of the antibody molecule), to obtain an aqueous antibody molecule-containing emulsion and then b) spray-drying the resulting aqueous antibody molecule-containing emulsion to obtain said dry microparticle with enhanced antibody molecule-sustained release performance. As a further example, herein is provided a method for enhancing the antibody molecule-sustained release performance of a dry microparticle, presenting a limited burst release upon injection and/or a better total release of the antibody molecule, said method comprising the steps of: a) adding cyclodextrin at an antibody molecule/cyclodextrin ratio (w/w) of from or from about 12:1 to or to about 7:6 and then about 0.5 to about 10.0% w/v of a polymer to an aqueous antibody molecule-containing solution (comprising about 5 to about 30% w/v (i.e. about 50 to about 300 mg/mL) of the antibody molecule and about 5 to 100 mM of the buffering agent), to obtain an aqueous antibody molecule-containing emulsion and then b) spray-drying the resulting aqueous antibody molecule-containing emulsion to obtain said dry microparticle with enhanced antibody molecule-sustained release performance. It is noted that should the microparticle comprise a surfactant, said surfactant is preferably added (during step a) or before step a)) in the aqueous antibody molecule-containing solution at about 0.05 to about 1.5% w/v. It is further noted that after the step of spray-drying, the dry microparticle may be further subjected to a step of freeze-drying.

In the context of the present disclosure as a whole, the antibody molecule is a complete antibody molecule having full length heavy and light chains, or an antigen-binding fragment thereof, for example selected from the group comprising or consisting of (but not limited to) a Fab, modified Fab, Fab′, modified Fab′, F(ab′)2, Fv, Fab-Fv, Fab-dsFv, Fab-Fv-Fv, scFv, Bis-scFv fragment, Fab linked to one or two scFvs or dsscFvs, such as BYbe® or a TRYbe®, diabody, tribody, triabody, tetrabody, minibody, single domain antibody, camelid antibody, Nanobody™ or VNAR fragment. The antibody molecule according to the invention can be a mono-, bi-, tri- or tetra-valent, bispecific, trispecific, tetraspecific or multispecific antibody molecule formed from antibodies or antibody fragments. Said antibody molecule can be present in the dry microparticle in a range from about 10 to about 30%, preferably from about 15 to about 30% and even more preferably from about 20 to about 30% such as 20, 21, 22, 23, 24, 25, 26, 27, 28, 29 or 30%. Before being dried, the antibody molecule is preferably present in an aqueous solution or in an emulsion at a concentration of or of about 50 mg/mL to or to about 300 mg/mL, preferably of or of about 50 mg/mL to or to about 200 mg/mL, or even preferably at a concentration of or of about 50 mg/mL to or to about 160 mg/mL, such as 50, 60, 70, 80, 90, 100, 110, 120, 130, 140, 150 or 160 mg/mL. Alternatively, before being dried, the antibody molecule is present in an aqueous solution or in an emulsion at a concentration of or of about 5 to or to about 30% w/v, or preferably at a concentration of or of about 5 to or to about 20% w/v, or even preferably at a concentration of or of about 5 to or to about 16% w/v, such as 5, 6, 7, 8, 9, 10, 11, 12, 13, 14, 15 or 16% w/v.

In the context of the present disclosure as a whole, the cyclodextrin is a member of the β-cyclodextrin family, such as HPβCD and SBEβCD. Alternatively, it can also be a member of the α-cyclodextrin family. It has been shown by the inventor that a specific range of antibody molecule/cyclodextrin ratio (w/w) was needed to obtain the best dry microparticle in term of stability, encapsulation, extraction and burst release. In the context of the present invention in its entirety, the antibody molecule/cyclodextrin ratio (w/w) is preferably from or from about 12:1 to or to about 7:6. Even preferably the antibody molecule/cyclodextrin ratio (w/w) is from or from about 10:1 to or to about 7:6, such as (about) 10:1, 9:1, 8:1, 7:1, 6:1, 5:1, 4:1, 3:1, 2:1, 3:2, 4:3, 5:4, 6:5 or 7:6. In the context of the present disclosure as a whole, the polymer is typically a biodegradable polymer preferably based on lactic acid or caprolactone. Exemplary of polymers that can be used according to the present invention are PLGA, PLA, PEG-PLGA or PCL. The polymer is added in the aqueous antibody molecule-containing solution at a concentration of about 0.5 to about 10.0% w/v, even preferably of about 1.0 to about 5.0% w/v, such as of about 1.0, 1.5, 2.0, 2.5, 3.0, 3.5, 4.0, 4.5 and 5.0% w/v. Said polymer will therefore be present in the dry microparticle in a range from about 50 to about 80%, such as 50, 55, 60, 65, 70, 75 or 80% w/w.

According to the present invention in its entirety, should a buffering agent be present, said buffering agent can be selected from the group comprising or consisting of (but not limited to) phosphate, acetate, citrate, arginine, trisaminomethane (TRIS), and histidine. Said buffering agent is preferably present in the aqueous antibody molecule-containing solution. The buffering agent is preferably present in an amount of from about 5 mM to about 100 mM of the buffering agent, and even preferably from about 10 mM to about 50 mM, such as about 10, 15, 20, 25, 30, 35, 40, 45 or 50 mM. Said buffering agent will therefore be present in the dry microparticle in a range from about 0.2 to about 4.0% w/w, such as 0.2, 0.3, 0.4, 0.5, 1.0, 1.5, 2.0, 2.5, 3.0, 3.5 or 4.0% w/w.

In the context of the whole disclosure, a surfactant may be present. Said surfactant is preferably a poloxamer such as poloxamer 407. The surfactant is preferably added in the aqueous antibody molecule-containing solution at a concentration of from or from about 0.05% to or to about 2.0% (w/v), more preferably from or from about 0.05% to or to about 1.5% (w/v) or even preferably from or from about 0.1% to or to about 1.0% (w/v), such as about 0.1, 0.2, 0.3, 0.4, 0.5, 0.6, 0.7, 0.8, 0.9 or 1.0% (w/v). Said polymer, if any, will therefore be present in the dry microparticle in a range from about 0.05 to about 4% w/w, such as 0.05, 0.1, 0.5, 1.0, 1.5, 2.0, 2.5, 3.0, 3.5 or 4.0% w/w. It is generally understood that in a water-in-oil emulsion the maximum volume of aqueous phase is 40% of the total volume (i.e. organic phase volume+aqueous phase volume). This corresponds to a aqueous phase: organic phase ratio (v/v) of not more than 6.7:10. In the context of the whole disclosure, the aqueous phase:organic phase ratio (v/v) ranges from 1/20 to 7/20, such as 1/20, 1/10, 3/20, 2/10, 5/20, 3/10 or 7/20.

Preferably, the aqueous antibody molecule-containing emulsion or the dry microparticle according to the invention as a whole does not comprise any sugar compound (e.g. does not comprise monosaccharide, disaccharide or any other polysaccharide, such as dextran or dextran-derived compound).

Another object of the present invention is a pharmaceutical composition comprising one or more of the dry microparticles according to the invention as a whole.

The invention also provides an article of manufacture, for pharmaceutical use, comprising a vial comprising any one or more of the above described dry microparticles, said microparticles comprising or consisting of an antibody molecule, a polymer, a cyclodextrin and optionally a buffering agent and/or a surfactant.

Alternatively, here described is an article of manufacture, for pharmaceutical use, comprising: 1) a first vial comprising any one or more of the above described dry microparticles, said microparticles comprising or consisting of an antibody molecule, a polymer, a cyclodextrin and optionally a buffering agent and/or a surfactant and 2) a second vial comprising a solvent for resuspension, should resuspension be needed.

The invention also provides a kit comprising; the dry microparticle(s) according to the present invention, an instruction manual and optionally a diluent (should the dry microparticle(s) be resuspended before use).

The dry microparticle(s) according to the invention may be stored for at least about 12 months to about 36 months. Under preferred storage conditions, before the first use, said microparticles are kept away from bright light (preferably in the dark), preferably at a temperature from about 2 to about 25° C.

Should the dry microparticle(s) of the invention be resuspended before use, resuspension is preferably performed under sterile condition, with a solvent, such as water or a saline solution (e.g. 0.9% w/v sodium chloride for injection) prior to use, i.e. prior to administration. The resuspended antibody composition should be administered preferably within one hour of resuspension.

The dry microparticle(s) according to the invention or the resuspended antibody composition according to the invention, is for use in therapy or diagnosis.

The dry microparticle(s) or the resuspended antibody composition(s) according to the invention are administered in a therapeutically effective amount. The precise therapeutically effective amount for a human subject may depend upon the severity of the disease state, the general health of the subject, the age, weight and gender of the subject, diet, time and frequency of administration, drug combination(s), reaction sensitivities and tolerance/response to therapy. This amount can be determined by routine experimentation and is within the judgement of the clinician. Generally, a therapeutically effective amount of antibody molecule will be from 0.01 mg/kg to 500 mg/kg, for example 0.1 mg/kg to 200 mg/kg or 1 mg/kg to 100 mg/kg.

The appropriate dosage will vary depending upon, for example, the particular antibody molecule to be employed, the subject treated, the mode of administration and the nature and severity of the condition being treated.

The dry microparticle(s) according to the present invention is/are administered preferably via the subcutaneous, intramuscular, intraarticular or intranasal route. Alternatively, the resuspended antibody composition(s) according to the present invention is/are administered by inhalation.

The following examples are provided to further illustrate the preparation of the pharmaceutical compositions, such as dry microparticles, of the invention. The scope of the invention shall not be construed as merely consisting of the following examples.

### FIGURES

FIG. 1: Production of Ab-loaded microparticles according to the invention.

FIG. 2: Release profile over time for the formulation comprising 67:33 mAb1/HPβCD.

FIG. 3: Comparison of the average mAb1 concentration in plasma over time for the SC, SOW and SD groups.

### EXAMPLES

1. Material

TABLE 1
Material used

| Material | Suppliers |
|---|---|
| mAb1 = IgG, 150 kDa, pI about 6.1 | UCB |
| fAb2 = Fab, 50 kDa, pI of about 9.5 | UCB |
| L-histidine | Sigma-Aldrich |
| Poloxamer 407 (LUTROL ® F127) | BASF |
| Ethyl acetate (EtAc) | Merck KGaA |
| Hydroxypropyl-beta-cyclodextrin (HPβCD) | TCI |
| Sulfobutyl ether beta-cyclodextrin (SBEβCD) | Ligand |
| (CAPTISOL ®) | |
| Gamma-cyclodextrin (γCD) | AppliChem |
| | GmbH |
| Poly (lactide-co-glycolide) copolymer, Resomer ® | Evonic |
| RG505 (ratio: 50:50) | Industries AG |

2. Methods

2.1 Preparation of Antibody-Containing Solutions:

The antibodies (Ab)-containing solutions were prepared from an initial formulation solution containing:
160 mg/mL of mAb1 in an aqueous solution comprising 30 mM histidine, 200 mM sorbitol, 60 mM sodium chloride, pH 5.6 or
50 mg/mL of fAb2 in an aqueous solution comprising 50 mM histidine, 125 mM sodium chloride, pH 6.0.

The formulation solutions were prepared by buffer exchange using appropriate centrifugal filter devices, such as the Amicon 15 30 KDa Mw Co membranes (Millipore, USA) or the VIVASPIN® 20 30 KDa membranes (Sartorius, Germany) or by using VIVAFLOW® 50 or 200 cassettes (Sartorious, Germany). The initial solutions were transferred into the appropriate formulation solutions by sequential dilution and concentration by centrifugation at 4000 g or by a gradual buffer exchange occurring through the passage of the different solutions into the cassette. The final antibody-containing solutions were filtered on 0.22 μm membranes using the STERITOP™ or STERIFLIP® filter Units (Millipore, USA) before further processing. The final antibody concentration, was 80 mg/mL (i.e. 8%) in 15 mM L-histidine pH 5.6 for mAb1 and in 50 mM L-histidine pH 6.0 for fAb2, in presence of 0.5% w/v of poloxamer 407 for both mAb1 and fAb2. The excipients, such as cyclodextrins or trehalose (from 100:0 to 20:80 w/w Antibody: cyclodextrin or trehalose ratio), were added before emulsification.

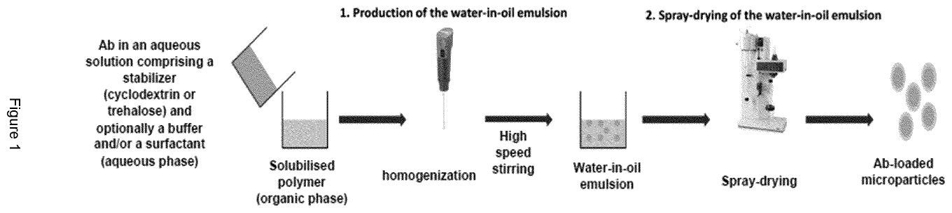

2.2 Encapsulation Process (FIG. 1)

The first step was the preparation of a water-in-oil (w/o) emulsion. In order to produce the w/o emulsion (e.g. 1:10 water/oil ratio), PLGA was firstly dissolved in ethyl acetate (PLGA concentration of 2.5% w/v). The w/o emulsion was obtained by pouring the antibody-containing solution into the organic phase under high speed stirring (using a T25 digital ULTRA-TURRAX® high speed homogenizer (IKA, Germany) equipped with a S25N—8 G dispersing tool set at 13,500 rpm during 1 minute. The emulsification step was performed at room temperature.

The second step was the spray-drying of the emulsion. This method is widely applied for converting aqueous or organic solutions, emulsions, dispersions and suspensions into a dry powder containing microparticles (alternatively named microspheres). A spray-dryer atomizes a liquid feed into fine droplets and evaporates the solvent or water by means of a hot drying gas. Process parameters such as inlet temperature, outlet temperature, atomization pressure, flow rate and aspiration were controlled during the process. The w/o emulsion obtained from the first step was spray-dried using a mini Spray-Dryer B-290® (Büchi, Switzerland) equipped with a two-fluid nozzle whose diameter value was 0.7 mm, under constant agitation, leading to dried microspheres (MS) (i.e. the dry microparticles). For each composition, the following parameters were kept constant with a gas spray flow at 600-800 L/h, an aspiration rate of 34 m³/h and a flow rate of 3.0 mL/min.

2.3. Protein Concentration—A280:

The “total Ab” assays were performed using UV spectrophotometry at 280 nm on a SpectraMax M5 microplate reader (Molecular Devices, USA).

2.4. Total Protein Assay by BCA (Bicinchoninic Acid) Colorimetric Assay:

The evaluation of Ab encapsulated inside MS was performed by total protein assay using the BCA method. The Pierce protocol “Microplate procedure” was followed. Before dosing the Ab inside MS, it was necessary to extract it from the MS. For this purpose, a known quantity of MS (10-20 mg) was placed in contact with 1 mL of NaOH 0.1N solution to dissolve the polymer and the protein. The working reagent was prepared by mixing 50 parts of BCA Reagent A (solution containing sodium carbonate, sodium bicarbonate, bicinchoninic acid and sodium tartrate in 0.1N sodium hydroxide) with 1 part of BCA Reagent B (solution containing 4% cupric sulphate). 25 μL of each standard or unknown sample was put into a microplate well. 200 μL of the working reagent was added to each well. After 30 seconds mixing on a plate shaker, the plate was covered and incubated at 37° C. for 30 minutes. The absorbance was measured at 562 nm on a SpectraMax M5 microplate reader (Molecular Devices, USA). A standard curve was prepared by plotting the average 562 nm measurement for each standard (gamma globulin or the Ab itself) vs. its concentration in μg/mL. This standard curve was used to determine the Ab concentration of each unknown sample. The DL (Drug Loading) was defined as the amount of Ab divided by the total amount of Ab and excipients and the EE (Encapsulation Efficiency) was calculated as the ratio between the obtained DL and the theoretical one.

2.5. Size Exclusion Chromatography (SEC):

SEC is one of the most commonly used analytical methods for the detection and quantification of both the HMWS (High Molecular Weight Species) and the LMWS (Low Molecular Weight Species). Insoluble aggregates are not considered to be measurable by SEC due to potential removal via filtration by the column or by the sample preparation for SEC.

For mAb1: SEC was performed on a Hewlett Packard Agilent 1200 high-performance liquid chromatography (Agilent Technologies, Germany) with a TSKgel G3000SWXL 7.8 mm×30.0 cm column (Tosoh Bioscience, Germany) and UV-detection at 280 nm. The flow rate was set at 1 mL/min and the injection volume was 50 μL. The mobile phase was a 0.2 M phosphate buffer solution (PBS), pH 7.0.

For fAb2: SEC was performed on a UPLC H class bio with an Acquity UPLC BEH200 4.6 mm×300 mm column coupled with an Acquity UPLC BEH200 guard column and UV-detection at 280 nm. The flow rate was set at 0.3 mL/min and the injection volume was 5 μL. The mobile phase was a 0.1 M phosphate buffer solution (PBS), pH 7.0 with 0.1M NaCl.

2.6. Extraction Efficiency

The extraction efficiency (ExE) referred to the percentage ratio between the amount of Ab extracted from the MS compared to the amount of Ab encapsulated that was determined by BCA, (see section 2.4 above). To extract the Ab from the MS, 10 mg of microparticles were dissolved in 500 μL of dichloromethane (DCM) or acetone (ACE) into NANOSEP® centrifugal devices with a porosity of 0.2 μm (Pall, Belgium) during approximately 2 h. The sample was centrifuged at 12,000 rpm for 5 minutes. The organic phase was removed and replaced by the same volume of fresh DCM or ACE. The sample was centrifuged at 12,000 rpm for 5 minutes again. This step was performed twice. The obtained precipitate was dried under vacuum for at least one hour and then solubilized in 500 μl of a phosphate buffer solution 200 mM pH 7.0. The samples obtained were then analysed by SEC in order to evaluate Ab stability after encapsulation. HMWS increase was calculated in comparison to the Ab reference that was the Ab solution obtained after the buffer exchange, before the encapsulation process. The highest is the ExE, the highest is the amount of encapsulated Ab that could be extracted, indicating that the Ab is still stable enough to be extracted and resolubilized. Besides, if the ExE is close to 100%, it means that the HMWS increase determined is highly representative of the state of all the Ab that was encapsulated.

2.7. Dissolution Study:

Dissolution profiles of Ab from Ab-loaded PLGA MS were evaluated by adding 1 mL of PBS buffered at pH 7.0 to 40 mg of MS in 2 mL tubes. The tubes were incubated at 37° C. and stirred at 600 rpm using a THERMOMIXER COMFORT® micro tubes mixer (Eppendorf AG, Germany). At a pre-determined time, samples were centrifuged for 15 minutes at 3000 g and the supernatant (1 mL) was collected and filtrated on 0.45 μm nylon ACRODISC® filter (Pall, France). The MS were suspended again in 1 mL of fresh PBS solution for further dissolution. The burst release was calculated as the percentage of Ab released after 24 hours. The burst release should be kept as low as possible in order to avoid issues such as drug concentrations near or above the toxic level or lack of efficacy (Huang and Brazel, 2001).

### Example 1

In this experiment, HPβCD was used as a stabilizing agent at different weight ratios to evaluate its influence on microspheres characteristics and the interest of using it for the limitation of HMWS formation. mAb1 was used for this example. The results are reported on Table 2.

Targeted EE (above 90%) were obtained for all formulations. While targeted DL (above 20% were obtained for all ratios except the 50:50 and 20:80 Ab/CD ratios, unacceptable ExE (below 80%) were obtained for the 94:6 Ab/CD ratio and for the formulation without any CD. Besides, increasing the percentage of HPβCD (i.e. decreasing the mAb1/stabilizer ratio) into the compositions led to an increase of the burst release. From the 50:50 mAb1/HPβCD ratio and lower ratios (as shown for 50:50 and 20:80 ratios), too high burst releases were obtained. Without any stabilizer, an unacceptable increase of HMWS was observed (above 13%). It was shown that the mAb1/HPβCD ratio also had an influence on mAb1 stability. Indeed, a significant limitation of mAb1 degradation could be observed from the 80:20 mAb1/HPβCD ratios and lower ratios (as shown below for 80:20, 67:33, 50:50 and 20:80 ratios). Finally, from the 80:20 mAb1/HPβCD ratio and lower ratios, a minimum of 89.4% of the mAb1 could be extracted, which indicates that the HMWS increases obtained at these ratios were representative of almost all the mAb1 that was encapsulated. In addition, from the 80:20 mAb1/HPβCD ratio and lower ratios, at the end of the dissolution test, a minimum of 90.8% of mAb1 was released, underlying that more than 90% of the total amount of encapsulated mAb was released.

TABLE 2
Influence of the mAb1/HPβCD ratio on DL, EE,
mAb1 stability, ExE, burst release and % of total
mAb released (average of experiments results)

| | | | HMWS | | Burst | Total mAb |
|---|---|---|---|---|---|---|
| | DL | EE | increase | ExE | release | released |
| Formulation | (%) | (%) | (%) | (%) | (%) | (%) * |
| Without | 22.8 | 96.4 | +13.2 | 67.1 | 39.4 | 71.9 |
| HPβCD | | | | | | |
| 94:6 | 22.3 | 94.1 | +12.8 | 75.9 | 33.4 | 82.3 |
| mAb1/HPβCD | | | | | | |
| 80:20 | 21.9 | 96.2 | +3.1 | 89.4 | 38.2 | 90.8 |
| mAb1/HPβCD | | | | | | |
| 67:33 | 20.7 | 96.6 | +0.5 | 96.7 | 37.8 | 98.3 |
| mAb1/HPβCD | | | | | | |
| 50:50 | 19.1 | 98.4 | +0.4 | 97.2 | 60.5 | 101.3 |
| mAb1/HPβCD | | | | | | |
| 20:80 | 12.4 | 100.6 | +0.4 | 103.2 | 87.4 | 104.6 |
| mAb1/HPβCD | | | | | | |

* Total mAb released at the end of the dissolution test
Particle sizes with a diameter of 5-10 μm (for Dv(0.5)) and of 20-50 μm (for Dv(0.9)) were obtained (Dv(0.5) = diameter below which lie 50% of the sample volumes and Dv(0.9) = diameter below which lie 90% of the sample volumes).

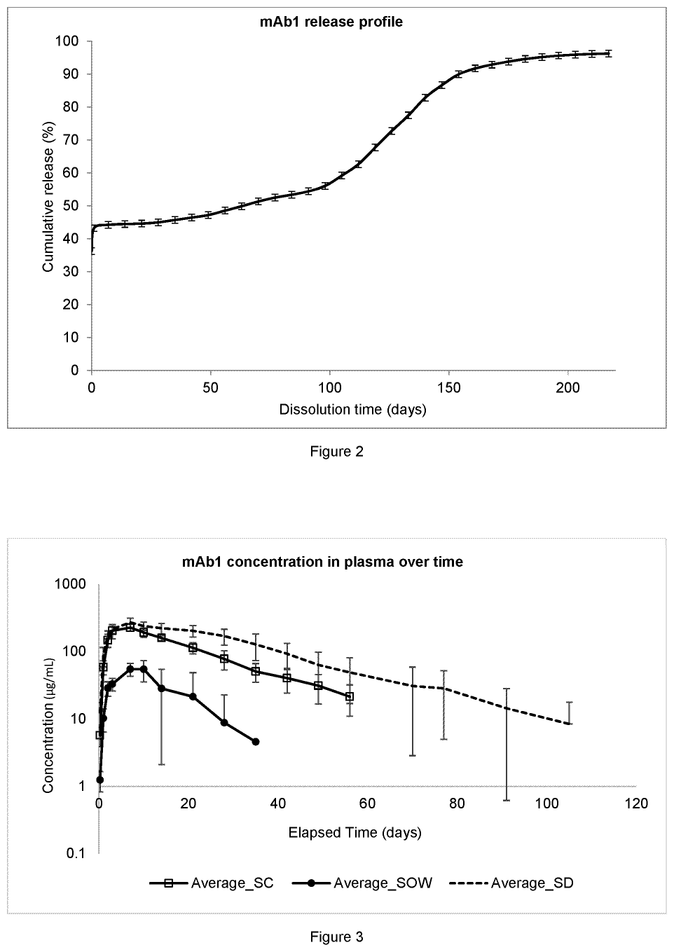

The typical triphasic release profiles for protein-loaded PLGA microparticles were observed (i.e. (i) an initial burst, (ii) a lag phase and (iii) a release phase; Diwan et al., 2001 and White et al., 2013), underlining no unexpected behavior for the formulation according to the invention. FIG. 2 shows the full release profile for the formulation comprising 67:33 mAb1/HPβCD.

To conclude, the addition of HPβCD at the most adequate Ab/HPβCD (67:33) ratio led to a limited HMWS increase (<1%) with a high DL (>20%), a targeted EE (≥90%) and an acceptable burst release (38%). The antibody/HPβCD (80:20) ratio led also to acceptable results, i.e. limited HMWS increase (<5%) with a high DL (>20%), a targeted EE ((≥90%) and an acceptable burst release (38%).

### Example 2

It was interesting to understand if two other cyclodextrins that are accepted for parenteral use in human (i.e. SBEβCD and γCD) were also suitable for Ab stabilization, and, if so, to compare the ratio needed for each cyclodextrin and the effect of their incorporation into the microspheres on the burst effect. Thus, encapsulation studies were performed with these two cyclodextrins.

Solubilization issues were observed when γCD was used. That was due to the presence of poloxamer 407 into the solution. Indeed, when only mAb1 and γCD were present, no problem of solubilization was observed. Consequently, it was necessary to perform the encapsulation process without using poloxamer 407 when γCD was used. Nevertheless, previous experiments showed that the removal of poloxamer 407 from the aqueous solution led to detrimental results in terms of emulsion stability (data not shown) and thus mAb1 release (only 80% of mAb1 release at the end of the study against 95-100% usually). Considering this, it was decided to evaluate only the 67:33 w/w mAb1/CD ratio for γCD.

It could be seen that, for all cyclodextrins, at all ratios studied, acceptable HMWS increases (lower than 5%) were observed (Table 3). However, at the 67:33 w/w mAb1/CD ratio, γCD led to a higher HMWS formation in comparison to the other cyclodextrins. Lower ExE were obtained for all ratios with SBEβCD and γCD, underlining that the HMWS increases observed were less representative of the encapsulated mAb1 in comparison to the use of HPβCD.

TABLE 3
Influence of the type of cyclodextrin on mAb1 stability
and ExE (average of experiments results)

| | HMWS increase (%) | | | ExE (%) | | |
|---|---|---|---|---|---|---|
| Formulation | HPβCD | SBEβCD | γCD | HPβCD | SBEβCD | γCD |
| 80:20 | +3.1 | +1.4 | NA | 89.4 | 85.1 | NA |
| mAb1/CD | | | | | | |
| 67:33 | +0.5 | +0.4 | +3.0 | 96.7 | 86.4 | 85.0 |
| mAb1/CD | | | | | | |
| 50:50 | +0.4 | +0.0 | NA | 97.2 | 88.3 | NA |
| mAb1/CD | | | | | | |

Considering the issues observed with γCD and the results obtained in terms of Ab stability, it was decided to evaluate only SBEβCD and HPβCD for the other parameters.

Targeted EE (above 85%) were obtained for both cyclodextrins, whatever the ratio mAb1/CD ratio studied (Table 4). The percentages of total mAb released at the end of the dissolution test were above 90% for all the ratios tested for both cyclodextrins. There was no significant influence of the type of cyclodextrin used on DL and EE. Differences of burst releases could be observed according to the type of cyclodextrin used, except for the 80:20 mAb1/CD ratio. Finally, at the most interesting ratio for mAb1 stability (67:33 mAb1/CD), HPβCD was the most suitable in terms of burst release.

TABLE 4
Influence of the type of cyclodextrin on DL, EE, burst release
and total mAb released (average of experiments results)

| | DL (%) | | EE (%) | | Burst release (%) | |
|---|---|---|---|---|---|---|
| Formulation | HPβCD | SBEβCD | HPβCD | SBEβCD | HPβCD | SBEβCD |
| 80:20 mAb1/CD | 21.9 | 22.5 | 96.2 | 99.6 | 38.2 | 37.5 |
| 67:33 mAb1/CD | 20.3 | 20.9 | 94.7 | 97.7 | 37.8 | 47.5 |
| 50:50 mAb1/CD | 19.1 | 19.2 | 98.4 | 98.9 | 60.5 | 55.0 |

| | Total mAb released at the end | |
|---|---|---|
| | of the dissolution test (%) | |
| Formulation | HPβCD | SBEβCD |
| 80:20 mAb1/CD | 90.8 | 93.5 |
| 67:33 mAb1/CD | 98.3 | 98.1 |
| 50:50 mAb1/CD | 101.3 | 95.7 |

To conclude, the use of γCD was not suitable for the purpose of this experiment. SBEβCD and HPβCD showed interesting results in terms of DL and EE. Besides, SBEβCD and HPβCD both allowed a limitation of HMWS increase. The use of HPβCD at the 67:33 w/w Ab/CD ratio was the most suitable considering the burst release. As in Example 1, the use of HPβCD at the 80:20 w/w Ab/CD ratio led also to acceptable results. Alternatively, very good results were also obtained with SBEβCD at the 80:20 w/w Ab/CD ratio. The 67:33 w/w Ab/CD ratio is also promising for both cyclodextrins, despite an increased burst release with SBEβCD.

### Example 3

In this experiment, a comparison between the use of HPβCD and trehalose, an excipient that is commonly used for Ab stabilization, was performed. First, a comparison of the two excipients at the same Ab/excipient w/w ratio was made. Then it was decided to also compare the two excipients based on the same Ab/excipient molar/molar ratio. Thus, formulations F1 and F2 have the same weight ratio of excipient regarding the Ab while F1 and F3 have the same molar ratio of excipient regarding the Ab.

At the same weight ratio, it seemed that HPβCD was more effective than trehalose to protect mAb1 against HMWS formation (Table 5). However, the values obtained in terms of HMWS increase were not greatly different for the two stabilizers (0.5% with HPβCD and 0.9% with trehalose).

Nevertheless, at the same molar ratio, mAb1 stability was greatly influenced by the stabilizer used. Thus, trehalose could not sufficiently prevent HMWS formation during the encapsulation process. Besides, the E×E values obtained for formulation F3 were lower than for the other formulations, confirming that this formulation led to more degradation of the mAb1.

TABLE 5
Influence of the type of stabilizer on mAb1 stability,
ExE, DL and EE (average of experiments results)

| | Molar | | | | | |
|---|---|---|---|---|---|---|
| | concen- | HMWS | | | | Total mAb |
| | trations | increase | ExE | DL | EE | released |
| Formulation | (mmol/L) | (%) | (%) | (%) | (%) | (%)* |
| 67:33 w/w | mAb1: 0.533 | 0.5 | 83.1 | 20.7 | 97.6 | 98.3 |
| mAb1/HPβCD | HPβCD: | | | | | |
| (F1) | 29.1 | | | | | |
| 67:33 w/w | mAb1: 0.533 | 0.9 | 83.6 | 21.1 | 99.5 | 97.9 |
| mAb1/trehalose | Trehalose: | | | | | |
| (F2) | 116.9 | | | | | |
| 89:11 w/w | mAb1: 0.533 | 6.3 | 77.4 | 22.7 | 98.5 | 89.7 |
| mAb1/trehalose | Trehalose: | | | | | |
| (F3) | 29.1 | | | | | |

*Total mAb released at the end of the dissolution test

Targeted EE (above 90%) were obtained for all formulations (Table 5). There was no significant influence of the type of stabilizer used on DL and EE. Similar burst releases were obtained for all formulations (data not shown). It could be seen that decreasing the amount of trehalose did not allow a decrease of the burst release (data not shown), contrary to what was previously observed with HPβCD (see Example 1).

To conclude, the interest of using HPβCD as a stabilizer over trehalose, a commonly used excipient for Ab stabilization, was demonstrated in this study. In particular, a lower molar amount of HPβCD than trehalose (4 times lower based on Table 5) was required to obtain Ab protection against HMWS formation.

### Example 4

This experiment aimed at applying the encapsulation process and more particularly the stabilization strategy developed for a mAb to a fAb in order to:
Evaluate the possibility of using the encapsulation process and the formulation strategy to different formats of antibodies,
Evaluate the influence of antibodies properties (size, degradation pathways) on microspheres characteristics.

For that purpose, a fAb molecule (named fAb2) was used. fAb2 is less prone to HMWS formation, contrary to mAb1 used in examples 1 to 3. The results of the study are reported in Tables 6 and 7. Without stabilizer, an increase of HMWS was observed but more limited than that observed for mAb1 (see Experiment 1). The fAb/HPβCD ratio had an influence on fAb stability. Formation of HMWS was almost completely suppressed from the 80:20 fAb/HPβCD ratio. For the 80:20 fAb/HPβCD ratio, almost 90% of the fAb could be extracted, which indicates that the HMWS increase obtained were representative of almost all the fAb that was encapsulated. A lower amount of HPβCD (80:20 fAb/CD) was sufficient to reduce HMWS formation compared to when the mAb was studied (67:33 fAb/CD). Very good results with regards to the reduction of HMWS formation were also obtained at 67:33 fAb/CD ratio.

Targeted EE (above 85%) were obtained for all formulations. Increasing the percentage of HPβCD into the formulation led to an increase of the burst release. The percentages of total mAb released at the end of the dissolution test were at or above 95% for all the ratios tested. Finally, higher burst releases than those obtained with the use of mAb were observed, underlining the influence of the size of the Ab (fAb2: 50 kDa vs. mAb1: 150 kDa) on the burst release.

TABLE 6
Influence of fAb/HPβCD ratio on mAb stability
and ExE (average of experiments results)

| | HMWS increase | ExE |
|---|---|---|
| Formulation | (%) | (%) |
| Without HPβCD | +4.7 | 77.8 |
| 94:6 fAb2/HPβCD | +2.3 | 88.9 |
| 80:20 fAb2/HPβCD | +0.1 | 88.4 |
| 67:33 fAb2/HPβCD | +0.2 | 81.1 |

TABLE 7
Influence of the fAb/HPβCD ratio on DL, EE
and burst release (average of experiments results)

| | | | | Total mAb |
|---|---|---|---|---|
| | DL | EE | Burst release | released |
| Formulation | (%) | (%) | (%) | (%)* |
| Without HPβCD | 23.6 | 100.8 | 48.6 | 88.3 |
| 94:6 w/w fAb2/HPβCD | 22.0 | 95.3 | 51.3 | 95.0 |
| 80:20 w/w fAb2/HPβCD | 22.0 | 99.3 | 57.2 | 97.8 |
| 67:33 w/w fAb2/HPβCD | 20.6 | 98.6 | 71.7 | 100.0 |

*Total mAb released at the end of the dissolution test

To conclude, the encapsulation process and the stabilization strategy could be successfully applied to a fAb. Although a burst release of above 50% was obtained with a fAb, the overall preliminary results are very promising. According to the antibody properties (size, mechanisms of degradation), an optimization of the Ab/HPβCD ratio should be performed. The skilled person would be able to optimize the formulation on the basis of the present description.

### Example 5—Role of the DL

In order to understand the influence of the DL on the Ab stabilization, their incorporation into the MS and on the burst effect, encapsulation studies were performed with two additional target DL: at 25% and 30%. As underlined in Table 8 below, although providing interesting results on EE and Ab stability, higher DL did not help with regards to the initial burst release. These results are promising but some fine tuning may be needed to improve the burst release.

TABLE 8
Influence of the theoretical DL on EE, burst release
and Ab stability (average of experiments results)

| | DL | EE | Burst release | HMWS increase |
|---|---|---|---|---|
| Formulation | (%) | (%) | (%) | (%) |
| DL_25 | 25.4 ± 0.3 | 92.0 ± 1.2 | 69.0 ± 6.1 | +1.1 ± 0.2 |
| DL_30 | 27.1 ± 0.3 | 84.4 ± 0.9 | 90.1 ± 2.9 | +1.3 ± 0.1 |

### Example 6

This experiment aimed at analyzing the in vivo effects of the dry microparticles according to the invention, in comparison with a typical dry-microparticles obtained from “solid-in oil-in water” (SOW) or a liquid subcutaneous (SC) formulation (as a control), when administered through one animal's flank. Experiments were performed with male Sprague-Dawley rats.

Animals were divided in 3 groups of 8 individuals:
Group 1 (8 rats; “SC” group; liquid formulation; immediate-release) received 30 mg/kg of mAb1, subcutaneously. The formulation contained 50 mg/mL of mAb in an aqueous solution composed of 30 mM L-histidine, 200 mM sorbitol and 60 mM sodium chloride.
Group 2 (6 rats; “SOW” group; dry microparticles resuspended in 0.9% w/v NaCl solution; sustained-release formulation). The targeted dose of mAb1 was 90 mg/kg. The formulation contained about 73.5% w PLGA (RG505), 17.1% w mAb1, 6.8% w trehalose, 1.7% w glycerol, 0.8% w histidine (as a buffering agent) and 0.02% w polysorbate 20.
Group 3 (8 rats; “SD” group; dry microparticles according to the invention resuspended in 0.9% w/v NaCl solution; sustained-release formulation). The targeted dose of mAb1 was 90 mg/kg. The formulation contained about 66.3% w PLGA (RG505), 21.2% w mAb1, 10.6% w HPβCD, 1.3% w poloxamer 407 and 0.6% w histidine (as a buffering agent).

For the three groups, each rat was administered the mAb1 formulation through one flank and a placebo formulation through the other flank. The placebo formulation for the SC group was a liquid solution, whereas the placebo formulation for the SOW and SD groups was a suspension of placebo microspheres.

For each group, samples were taken as follow: 6 h, 24 h, 48 h, day 3, day 7, day 10, day 14 and once a week until no more mAb1 was detected into the plasma samples.

The doses effectively administered were as follow:
SOW group: 42.1-43.3 mg/kg (1.4-fold increase compared to the SC group),
SD group: 77.4-81.1 mg/kg (2.7-fold increase compared to the SC group).

The mAb concentration in plasma over time was determined by ELISA.

Results are presented in FIG. 3 for all the formulations.
SC group: A typical profile for SC administration was observed for all of the animals belonging to this group. mAb1 was still detected in plasma up to 50+ days. Immunogenicity was suspected for one animal.
SOW group: mAb1 was detected in plasma up to 40+ days in this group. However, the profiles were very disparate, especially after 10 days from administration. Immunogenicity was suspected for most of the animals.
SD group: Contrary to the other groups, mAb1 was detected in plasma for more than 100 days. The profile was quite similar for most of the animals. Although not fully comparable because of the difference of dosing between each group, the dry microparticles according to the invention clearly allow a much longer delivery time, doubling the release time compared to the SOW group.

PK parameters were also evaluated (AUCINF_D_obs, Cmax, t½ and tₘₐₓ)(Table 9). The points seemingly impacted by immunogenicity were removed for calculating these parameters. In addition, the data were normalized to the dose effectively administered.

TABLE 9
Influence of the formulations on PK parameters
(average of experiments results)

| | SC | SOW | SD |
|---|---|---|---|
| AUCINF_D_obs | 0.1935 ± | 0.0266 ± | 0.1292 ± |
| (day · μg/ml/μg/kg) | 0.0281 | 0.0116 | 0.0334 |
| Bioavailability (%) | 100* | 13 | 68 |
| Cₘₐₓ (μg/mL) | 235.01 ± | — | 274.29 ± |
| | 33.86 | | 39.00 |
| t_{1/2} (days) | 14.44 ± | 8.13 ± | 17.37 ± |
| | 4.20 | 1.66 | 4.30 |
| tₘₐₓ (days) | 6.00 ± | 8.50 ± | 8.25 ± |
| | 1.85 | 1.64 | 5.34 |

*Bioavailability for SC set at 100%

As it can be observed from Table 9, the best value in comparison with SC were obtained with the SD group. It is noted that:
There is no significant increase of the Cₘₐₓ with dose increase.
The bioavailability is much higher for the SD group than for the SOW group, together with a more than twice higher T_{1/2}.
An increase of tₘₐₓ was observed in both SOW and SD groups.

### OVERALL CONCLUSION

In view of the results obtained in examples 1 to 5, the inventors have demonstrated that cyclodextrins, in particular HPβCD, and at a lesser extend SBEβCD, can be successfully used to stabilized antibodies in spray-dried formulations, whatever the antibody formats (e.g. mAb or fAb) and their pI. In particular, it was shown that antibody/stabilizer ratios of between 12:1 to 7:6 overall improve the performance of spray dried formulation. It was also shown that a lower molar amount of cyclodextrin (such as HPβCD) than trehalose (a standard stabilizer) was required to obtain antibody protection against HMWS formation (4 to 7 times lower). Example 6 confirmed the promising results of examples 1 to 5, demonstrating that the dry microparticles of the invention were effectively able not only to greatly improve the bioavailability compared to a standard SOW formulation but to also improve the slow-release profile of antibody-containing dry microparticles.

### REFERENCES

1. Wang et al., Stabilization and encapsulation of human immunoglobulin G into biodegradable microspheres. J Colloid Interface Sci. 2004; 271(1):92-101.
2. Giunchedi et al., Emulsion Spray-Drying for the Preparation of Albumin-Loaded PLGA Microspheres. Drug Dev Ind Pharm. 2001; 27(7):745-50.
3. Moussa et al., Immunogenicity of Therapeutic Protein Aggregates. J. Pharm. Sci. 2016; 105(2):417-30.
4. Pai et al., Poly(ethylene glycol)-modified proteins: implications for poly(lactide-co-glycolide)-based microsphere delivery, The AAPS Journal, 2009; 11(1):88-98.
5. Servo et al., Inhibition of agitation-induced aggregation of an IgG-antibody by hydroxypropyl-beta-cyclodextrin. J. Pharm. Sci. 2010; 99(3):1193-1206.
6. U.S. Pat. No. 5,997,856.
7. Johansen et al., Improving stability and release kinetics of microencapsulated tetanus toxoid by co-encapsulation of additives, Pharmaceutical Research, 1998; 15(7):1103-1110.
8. Han et al., Bioerodable PLGA-Based Microparticles for Producing Sustained-Release Drug Formulations and Strategies for Improving Drug Loading, Frontiers in Pharmacology, 2016; 7: article 185.
9. Diwan and Park., Pegylation enhances protein stability during encapsulation in PLGA microspheres. J Control Release. 2001; 73(2-3):233-44
10. White et al., Accelerating protein release from microparticles for regenerative medicine applications. Mater Sci Eng C [Internet]. Elsevier B.V.; 2013; 33(5):2578-83.
11. Verma et al., 1998, Antibody engineering: comparison of bacterial, yeast, insect and mammalian expression systems. Journal of Immunological Methods, 216, 165-181
12. Huang, X., Brazel, C. S. On the importance and mechanisms of burst release in matrix-controlled drug delivery systems. J. Control Release. 2001; 73 (2-3):121-36.

1. A dry microparticle comprising an antibody molecule, a polymer, and cyclodextrin.

2. An aqueous antibody molecule-containing emulsion comprising an antibody molecule, a polymer, and cyclodextrin.

3. The dry microparticle according to claim 1, wherein the cyclodextrin is a member of the β-cyclodextrin family.

4. The dry microparticle according to claim 1, wherein the antibody molecule is a complete antibody molecule having full length heavy and light chains, or an antigen-binding fragment thereof.

5. The dry microparticle according to claim 1, wherein the antibody molecule/cyclodextrin ratio (w/w) is from 12:1 to 7:6.

6. The dry microparticle according to claim 1, wherein the polymer is selected from the group consisting of PLGA, PLA, PEG-PLGA and PCL.

7. The dry microparticle according to claim 1, which further comprises a buffering agent.

8. The dry microparticle according to claim 7, wherein the buffering agent is selected from the group consisting of phosphate, acetate, citrate, arginine, TRIS, and histidine.

9. The dry microparticle according claim 1, which further comprises a surfactant.

10. The dry microparticle according to claim 9, wherein the surfactant is poloxamer 407.

11. The dry microparticle according to claim 1, which comprises about 10 to 30% weight (w/w) of the antibody molecule, about 50 to 80% (w/w) of the polymer, the cyclodextrin in an antibody molecule/cyclodextrin ratio (w/w) of from or from about 12:1 to or to about 7:6 and optionally about 0.2 to 4% (w/w) of the buffering agent and/or about 0.05 to 4.0% (w/w) of the surfactant.

12. (canceled)

13. A method for producing the dry microparticle according to claim 1, comprising the steps of:
a) adding cyclodextrin to an aqueous antibody molecule-containing solution to obtain an aqueous phase,
b) solubilising a polymer in a solvent, to obtain an organic phase,
c) adding the aqueous phase of step a) to the organic phase of step b) to obtain an aqueous antibody molecule-containing emulsion and then,
d) spray-drying, and optionally further freeze-drying, the resulting aqueous antibody molecule-containing emulsion to obtain the dry microparticle.

14. A method for stabilizing an antibody molecule in a dry microparticle comprising the steps of adding cyclodextrin and a solubilised polymer to an aqueous antibody molecule-containing solution to obtain an aqueous antibody molecule-containing emulsion and then spray-drying the resulting aqueous antibody molecule-containing emulsion, and optionally further freeze-drying it in order to obtain the stabilised antibody molecule in said dry microparticle.

15. A process for obtaining the dry microparticle according to claim 1, comprising the steps of:
a. adding cyclodextrin to an aqueous antibody molecule-containing solution to obtain a first composition,
b. combining the first composition of step a. with a polymer, wherein said polymer is solubilized, to obtain a second composition,
c. homogenizing the second composition of step b. to obtain a water-in-oil emulsion,
d. spray-drying the water-in-oil emulsion of step c. to obtain said dry microparticle, and
e. optionally freeze-drying the dry microparticle of step d. to obtain the final dry microparticle.

16. (canceled)

17. A pharmaceutical composition comprising one or more of the dry microparticles according to claim 1.

18. The aqueous antibody molecule-containing emulsion according to claim 2, wherein the cyclodextrin is a member of the β-cyclodextrin family.

19. The aqueous antibody molecule-containing emulsion according to claim 2, wherein the antibody molecule is a complete antibody molecule having full length heavy and light chains, or an antigen-binding fragment thereof.

20. The aqueous antibody molecule-containing emulsion according to claim 2, wherein the antibody molecule/cyclodextrin ratio (w/w) is from 12:1 to 7:6.

21. The aqueous antibody molecule-containing emulsion according to claim 2, wherein the polymer is selected from the group consisting of PLGA, PLA, PEG-PLGA and PCL.

22. The aqueous antibody molecule-containing emulsion according to claim 2, which further comprises a buffering agent, a surfactant, or a combination thereof.
